(19) United States
(12) Patent Application Publication
(10) Pub. No.: US 2023/0116083 A1
(43) Pub. Date: Apr. 13, 2023

(54) UP-SCALED PRODUCTION OF MICROGLIA-LIKE/-PRECURSOR CELLS AND MACROPHAGE CELLS USING MESH MACROCARRIERS

(71) Applicants: LIFE & BRAIN GMBH, Bonn (DE); RHEINISCHE FRIEDRICH-WILHELMS-UNIVERSITÄT BONN, Bonn (DE)

(72) Inventors: Mona Ann MATHEWS-AJENDRA, Bonn (DE); Simone HAUPT, Köln (DE); Oliver BRÜSTLE, Bonn (DE)

(21) Appl. No.: 17/908,857

(22) PCT Filed: Mar. 10, 2021
(86) PCT No.: PCT/EP2021/056037

§ 371 Date: Sep. 1, 2022

(30) Foreign Application Priority Data
Mar. 10, 2020 (EP) 20162230.5

(51) Int. Cl. C12N 5/071; C12M 1/12; C12N 5/079; C12N 5/0786

(52) U.S. Cl. CPC C12N 5/0697; C12M 25/02; C12M 25/14; C12N 5/0622; C12N 5/0645; C12N 2506/45; C12N 2531/00; C12N 2533/90

(57) ABSTRACT

The present invention relates to methods allowing adherence and outgrowth of embryoid bodies (EBs) using macrocarriers. The methods of the invention are useful for an up-scaled production of myeloid cells, such as macrophage- and microglia-like/-precursor cells, in a bioreactor system. The invention further relates to microglia-like cells or microglial precursor cells obtainable by these methods that are cryopreservable. The invention also concerns a porous macrocarrier coated with a material facilitating cell adherence.

### PUBLIC FUNDING

The project leading to this application has received funding from the Innovative Medicines Initiative 2 Joint Undertaking under grant agreement n° 115976. This Joint Undertaking receives the support from the European Union’s Horizon 2020 research and innovation programme and EFPIA.

### FIELD OF THE INVENTION

The present invention relates to methods allowing adherence and outgrowth of embryoid bodies (EBs) using macrocarriers. The methods of the invention are useful for an up-scaled production of myeloid cells, such as macrophage- and microglia-like/-precursor cells, in a bioreactor system. The invention further relates to microglia-like cells or microglial precursor cells obtainable by these methods. The invention also concerns a porous macrocarrier coated with a material facilitating cell adherence.

### BACKGROUND OF THE INVENTION

### Development of Microglial and Peripheral Macrophages

Microglia are the residential professional immune response cells exclusively found in the central nervous system (CNS). Although they bear similarities to their peripheral counterparts such as blood monocytes and peripheral tissue macrophages, microglia are derived from a spatially and temporally distinct progenitor population during primitive hematopoiesis (reviewed by Prinz and Priller, 2014). Primitive hematopoietic progenitors arise on blood islands in extra-embryonic tissue, from where the derived immature microglia infiltrate the embryo including fetal liver and take up lifetime residence in the CNS (Ginhoux et al., 2010; Kierdorf et al., 2013). Once in the CNS, microglia acquire a tissue-specific phenotype, which solidifies the microglial identity and broadens their distinction from peripheral immune cells (Bennett et al., 2016; Galatro et al., 2017; Gosselin et al., 2014, 2017).

In a second developmental step, definitive hematopoiesis occurs in the fetal liver, and postnatally in the bone marrow, giving rise to myeloid precursors that distribute throughout the body including the peripheral nervous system. Here, peripheral myeloid cells such as blood monocytes and macrophages among others contribute towards immune responses in the body (reviewed by Prinz and Priller, 2014).

### Sources of Microglia and Macrophages

Early microglial research made use of primary microglia from animal models, which were immortalized using SV40 transduction (Blasi et al., 1990). Alternatively, human microglia isolated from post-mortem or fetal tissues were transduced to obtain immortalized cell lines (Janabi et al., 1995). Human immortalized microglia were advantageous for large-scale molecular analysis of human specific diseases, since the cells could be infinitely expanded. However, immortalization and extended culturing of these cells are known to promote accumulation of mutations and epigenetic changes that cause artifacts in the cellular phenotype.

On the other hand, monocyte-derived macrophages are typically obtained by differentiating blood monocytes into an adherent macrophage-like population. While access to and acquisition of human blood is considered standard medical procedure, the macrophages needed for in vitro analysis can only be obtained from a subset of cells found in blood, namely blood monocytes. The process of obtaining blood monocytes requires the isolation of a total population of peripheral blood mononuclear cells (PBMCs) consisting of lymphocytes (such as T cells, B cells and NK cells), monocytes and possibly dendritic cells. Monocytes typically make up 10 - 20% of PBMCs and therefore represent a limited resource. In addition, high variations in quantity and quality of monocytes introduce variability in data (Kleiveland, 2015). Another common source of human in vitro cultured macrophages are immortalized cells lines such as THP1, obtained from a patient with acute myeloid leukemia. However, as common with many immortalized cell lines, the accumulation of mutations over several passages of culturing lessens the quality of these cell lines as a stable source to represent in vivo macrophages (Bosshart and Heinzelmann, 2016).

More recently, the availability of human induced pluripotent stem cells (hiPSCs) has opened up the possibility to derive hematopoietic cells in vitro, and several protocols for generating microglia-like and macrophage-like cells have been developed, as described below.

### Generation of Microglial Precursors, Microglia-Like and Peripheral Macrophage-Like Cells

Current progress in generation of in-vitro human microglia-like cells has benefited from studies that identified ontogeny and developmental process of these cells.

Protocols aiming to generate microglial progenitors from human induced pluripotent stem cells (hiPSCs) typically transition through an embryoid body (EB) phase (Abud et al., 2017; Haenseler et al., 2017; Muffat et al., 2016). In the above-mentioned publications, the generated EBs are induced almost exclusively towards primitive hematopoiesis using a wide combination of known small molecules. Alternatively, iPSCs can also be directed towards myeloid differentiation in a monolayer (Douvaras et al., 2017a; Pandya et al., 2017). Nevertheless, the cells were again almost exclusively differentiated towards the primitive hematopoietic fate. While some of these protocols yield relatively high cell numbers due to the focused induction, the derived cells remain immature upon harvesting. Their maturation into microglia-like cells requires additional steps including extended culturing in specific combinations of growth factors or co-culturing with neurons (Abud et al., 2017; Garcia-Reitboeck et al., 2018; Haenseler et al., 2017; Muffat et al., 2016; Takata et al., 2017). The importance of additional maturation steps that mimic an in vivo-like neural environment is highlighted by recent studies that have identified the influence of tissue environment on the identity of microglia (Galatro et al., 2017; Gosselin et al., 2017). Although production of higher numbers of microglial progenitors could be achieved by implementing large culture flask formats, the requirement of additional maturation steps precludes true scalability of the obtained cell type. A previous patent application from the applicant on the differentiation of microglia-like cells from iPSC in a neural environment provides the necessary microenvironment for the generated microglial precursors to mature towards a more microglial-like phenotype (WO 2010/125110 A1, also published as US 2012/0107898 A1; the content of both patent applications is hereby incorporated by reference). While the protocol described in WO 2010/125110 A1 achieves the purpose of generating ready-to-use microglia with the help of co-differentiating neural cell types within the same differentiation and therefore not requiring further maturation, the issue of limited number of cells obtained remains. Furthermore, this and other published protocols that currently represent the state-of-art in obtaining microglia-like cells are performed under 2D and static cell culture conditions (Abud et al., 2017; Douvaras et al., 2017b; Haenseler et al., 2017; Pandya et al., 2017; Takata et al., 2017; van Wilgenburg et al., 2013), which implies significant expenses for material and personnel.

In the case of peripheral immune cells, recent research highlighting the ontogeny of blood monocytes and macrophages also provides the opportunity for in vitro cell production (Medvinsky et al., 2011). Implementation of specific WNT modulating small molecules allows for the differentiation of hiPSC towards a definitive hematopoietic population (Ditadi and Sturgeon, 2016; Sturgeon et al., 2014). These break-throughs provide a strong basis for the development of robust monocyte-derived macrophages from hiPSC for in vitro disease modeling. However, as described for production of hiPSC-derived microglia, true scalability in terms of efficient growth area to volume ratio with implementation of minimal material costs and personnel time is still lacking. As the differentiation of hiPSC-derived macrophages may also typically transition through an EB stage, the use of a compatible carrier structure that supports EB adherence and outgrowth in large scale remains necessary.

### Macrocarrier-Based Bioreactor Culture for Scalable Production of iPSC-Derived Microglia

Current state-of-art protocols for iPSC differentiation into somatic cell types in bioreactors primarily involve culturing the cells as aggregates or seeded on microcarriers composed of various bio-compatible material and coatings such as Matrigel™ or extracellular matrices (reviewed in Badenes et al., 2016). The current commercially available carriers are in the micrometer range and require the seeding of single cells. These described carriers do not support the seeding or culturing of embryoid bodies (EBs), as required for microglial differentiation, which are typically spherical and ranging in size from 100 to 400 µm. International Patent Application WO 2009/116951 describes the use of matrix-covered microcarriers with a generally spherical shape in the range of 20 - 250 µm or with a generally cylindrical shape with the longest dimension ranging from 20 µm to 2000 µm to seed EBs, which facilitates adherence of EBs. However, these microcarriers lack the infrastructure in terms of growth area required to provide support of EB outgrowth, which is typical of microglial production protocols.

### TECHNICAL PROBLEMS UNDERLYING THE PRESENT INVENTION

As explained above, true scalability of the production of microglial precursors, microglia/-like and macrophage-like cells remained a problem in the prior art. In particular, previous protocols for the production of microglia involved significant expenses for material and personnel.

The present inventors have now established a novel protocol for the production of human iPSC-derived macrophage- and microglia-like/precursor cells. This protocol involves the use of a mesh membrane, typically used for filtration, as a macrocarrier to generate human iPSC-derived macrophage- and microglia-like/-precursor cell types in dynamic culture. The structure of the mesh provides support for firm adherence and outgrowth of embryoid bodies in 3D space and facilitates better induction and expansion of cystic structures that are a typical feature of hematopoiesis during generation of the microglial progenitors. In addition, the porous space allows for better gaseous and nutrient diffusion. Furthermore, the seeded meshes can be placed in a non-tissue culture format to ease the accessibility and harvest of the derived macrophage-, microglia-like / -precursor cells from the supernatant. The non-tissue culture format and dynamic culture also prevent the released cells from adhering to the plate or meshes, therefore easing the harvesting procedure further, with a high degree of purity. The novel protocol is suitable for long-term cell differentiation.

Purity can be further improved by passing the cell suspension through cell strainer and furthermore, by fluorescence-activated cell sorting (FACS) or magnetic-activated cell sorting (MACS).

The microglia-like cells and microglial precursor cells produced by the methods of the invention are surprisingly better suited for cryopreservation than corresponding cells of the prior art.

The above overview does not necessarily describe all problems solved and advantages achieved by the present invention.

### SUMMARY OF THE INVENTION

In a first aspect the present invention relates to a method for providing structural support to embryoid bodies for adherence and outgrowth, said method comprising the following steps:
(a) providing embryoid bodies;
(b) placing the embryoid bodies onto a macrocarrier, thereby obtaining a macrocarrier with adherent embryoid bodies; and
(c) culturing the macrocarrier with the adherent embryoid bodies of step (b) in cell-culture medium.

In a second aspect, the present invention relates to a method for producing microglia-like cells and/or microglial precursor cells comprising the following steps:
(i) carrying out the method according to the first aspect;
(ii) continuing cultivation of the macrocarrier with the adherent embryoid bodies in cell-culture medium until microglial precursor cells and/or microglia-like cells are released into the medium; and
(iii) optionally harvesting and/or cryopreserving the microglial precursor cells and/or microglia-like cells released into the medium.

In a third aspect the present invention relates to a microglia-like cell or microglial precursor cell obtainable by the method according to the second aspect.

In a fourth aspect the present invention relates to a use of a macrocarrier as structural support for adherence and outgrowth of embryoid bodies.

In a fifth aspect the present invention relates to a macrocarrier coated with a material facilitating cell adherence, wherein said macrocarrier is porous.

In a sixth aspect the present invention relates to a microglia-like cell or microglial precursor cell population, wherein said population is cryopreservable.

This summary of the invention does not necessarily describe all features of the present invention. Other embodiments will become apparent from a review of the ensuing detailed description.

### BRIEF DESCRIPTION OF THE FIGURES

FIG. 1. Schematic representation of microglia differentiation from induced pluripotent stem cells. (1) Embryoid bodies are generated in suspension culture following published protocols or WO 2010/125110 A1 or as described in current patent application. (2) After specification, EBs are seeded on to pre-treated mesh membranes, which serve as macrocarriers, at a density of up to 50 EBs per square centimeter in non-tissue culture formats. EBs adhere within 24 hours of inoculation and further give rise to a mixed progenitor layer of cells, which grow outwards over the surface and between the pores of the macrocarriers. (3) Once differentiated, microglia detach from the outgrowth into the dynamically moving supernatant from which they can be easily and efficiently harvested for experimentation.

FIG. 2. Image of a macrocarrier of the invention with an adherent embryoid body. (A): Embryoid bodies (EBs) adhere onto the macrocarriers coated with extracellular matrices and give rise to an outgrowth of progenitor cells, which grows outward over the mesh surface. Scale bar: 100 µm (B): The inset shows that the outgrowths of cells grow along the fibers (dotted arrows) and between the mesh pores (solid line arrows). Scale bar: 100 µm

FIG. 3. Comparison of commercially available macrocarriers (A, B) with the macrocarrier of the invention (C). Embryoid bodies (EBs) loosely adhere to Bionoc II™ (Cesco Bioengineering) and Fibra-Cel® (Eppendorf, Germany) carriers. Loose adherence does not facilitate the outgrowth of progenitors along the carriers (FIGS. 3A and 3B). EBs seeded on mesh membrane carriers adhere firmly within 24 hours and spread out along the mesh membrane, consequently giving rise to an outgrowth of progenitor cells (FIG. 3C). Scale bar: 500 µm

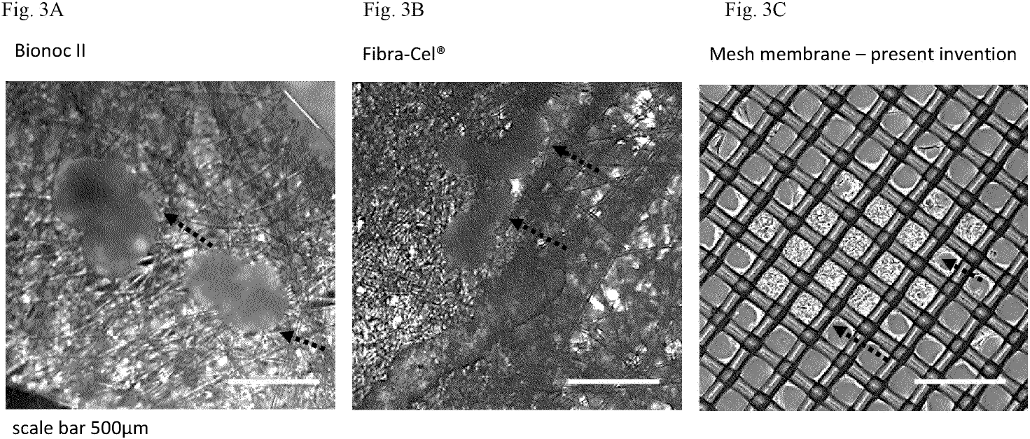

FIG. 4: Photograph of mesh membrane floating in cell culture medium. (A): (Top) Example side-view image shows the growth of three-dimensional cystic structures from the macrocarrier of the invention (here: a mesh membrane), as expected to arise during hematopoietic differentiation over time. (B): (Inset, bottom) the cystic structures are anchored to and supported by the mesh membrane to expand in three-dimensional space. The mesh membranes float freely in suspension culture, while cystic structures remain anchored on the mesh membrane.

FIG. 5: Comparison of the effect of different pore sizes and the effect of ECM coating on the adherence and outgrowth of embryoid bodies, 24 hours post inoculation. The three images in the top row show uncoated macrocarriers; the three images in the bottom row show ECM-coated macrocarriers. The two macrocarriers in the left column have 5 µm pore size; the two macrocarriers in the middle column have 60 µm pore size; and the two macrocarriers in the right column have 180 µm pore size. Seeded EBs poorly adhere to non-coated macrocarriers, while coated carriers in all pore sizes shown facilitate adherence of EBs and respective outgrowth of progenitors. Macrocarriers are shown 24 hours after inoculation. Mesh membranes to be used as macrocarriers (Nylon; 47 mm diameter) were obtained from MERCK Millipore, Germany. D indicates thickness of the membranes; P indicates porosity of the membranes according the data sheet provided by the manufacturer. Scale bar: 100 µm

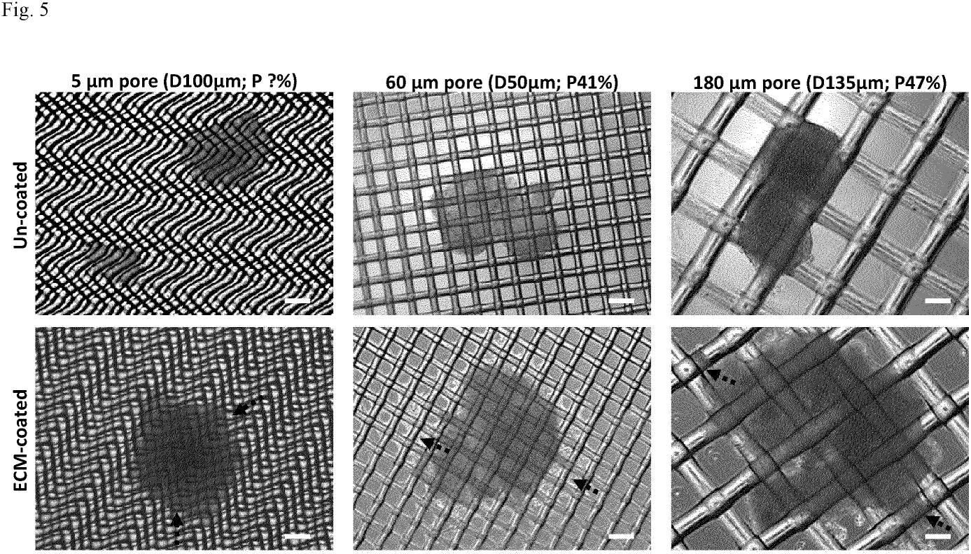

FIG. 6: Detailed differentiation protocol from embryoid bodies (EBs) to microglia-like cells/microglia-precursor cells released into supernatant. The diagram shows an exemplary protocol that was used in the experimental part of the application and that is well-suited for practicing the invention. The left part shows the media used in a three-day protocol for the differentiation of EBs in suspension culture. The middle part shows the medium used after placing the EBs on the macrocarrier (at least 24 hours for firm adherence). The right part shows the medium used in dynamic suspension culture for differention to microglia-like/-precursor cells.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (ILTPAC Recommendations)”, Leuenberger, H.G.W, Nagel, B. and Kolbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents (for example: patents, patent applications, scientific publications, manufacturer’s specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference ”. In the event of a conflict between the definitions or teachings of such incorporated references and definitions or teachings recited in the present specification, the text of the present specification takes precedence.

The term “embryoid bodies” as used herein (abbreviated as: EBs) refers to aggregates of cells derived from pluripotent stem cells. Embryoid bodies (embryoid body) are generally comprised of a large variety of differentiated cell types. Cell aggregation is for example imposed by hanging drop or other methods that prevent cells from adhering to a surface, thus allowing the embryoid bodies to form their typical colony growth. Upon aggregation, differentiation is typically initiated and the cells begin to a limited extent to recapitulate embryonic development. In the context of the present invention, the term “embryoid bodies” covers embryoid bodies irrespective of the method by which they were produced. Thus, the term “embryoid bodies” covers dynamically/bioreactor-generated embryoid bodies as well as the standard static-generated EBs. The term “embryoid bodies” covers EBs from any mammalian species, but preferably covers EBs from rats, mice or primates (including humans) and most preferably refers to human EBs. In one embodiment, the “embryoid bodies” are derived from human embryonic stem cells. In a preferred embodiment the “embryoid bodies” are derived from human induced pluripotent stem cells (hiPSCs).

In accordance with the present invention, the term “microglial precursor cells” relates to a population of cells comprising partially differentiated cells, derived from myeloid precursor cells and capable of further differentiating or maturing into microglial/-like cells. Myeloid precursor cells are characterized by the expression of the transcription factor PU. 1 (Iwasaki et al., 2005). Microglia are further characterized by a ramified morphology with processes interdigitating with other glial cells and neurons and in surveying their local environment (Ransohoff and Perry, 2009). Furthermore, upon transplantation of microglial precursor cells into tissues comprising neurons and astrocytes, they integrate into these tissues as microglia (Napoli, 2008; Tsuchiya et al., 2005). In addition, upon contact of microglial precursors with neurons and/or astrocytes or addition of growth factors such as macrophage colony-stimulating factor, they can transform into microglia (Liu et al., 1994).The population of “microglial precursor cells” may comprise cells at different stages of differentiation between myeloid precursor cells and microglia/-like cells. Thus, also fully differentiated microglial cells may be comprised in the population of microglial precursor cells. Non-differentiated stem cells as well as neural or mesenchymal stem cells are not comprised in the term “microglial precursor cells”.

As used herein, a “microglia-like cell” is a cell that was derived in vitro from pluripotent stem cells (embryonic stem cells or preferably induced pluripotent stem cells (iPSCs), more preferably from human induced pluripotent stem cells (hiPSCs) and that resembles a microglia cell isolated from an in vivo source of the same species. In particular, the expression signatures of “microglia-like cells” resemble those of purified human fetal or adult microglia maintained in the same culture conditions and recapitulate the consensus signature of microglia compared to other macrophages. They perform the functions of professional phagocytes, are positive for microglial markers such as TMEM119, and react to canonical stimuli.

“Microglia-like cells” prepared according to the second aspect of the present invention (see above, section “Summary of the Invention”, and see below, section “Embodiments of the Invention”) are indistinguishable from naturally occurring “microglia cells”. Consequently, all aspects and embodiments of the present invention referring to “microglia-like cells” can also be worded as referring to “microglia cells”.

Classically, the term “macrophage” refers to a set of terminally differentiated cells with low proliferation capacity that have different names according to their localization. Despite the common name, macrophages do not represent a homogeneous population and can originate from two distinct embryological sources. The first wave of macrophages is generated during early embryogenesis and is derived from Yolk Sac progenitors, giving rise to physiological tissue macrophages (named microglia, Langerhans cells, Kupffer cells and alveolar macrophages). On the other hand, the second wave of macrophages is derived from Hematopoietic Stem Cells (HSCs) that become circulating blood monocytes, which after migration to peripheral tissues differentiate into monocyte-derived macrophages (MDMs) (Galvão-Lima et al., 2017).

As used herein, the expression “macrophage- and microglia-like/-precursor cells” refers to a cell population comprising macrophages and/or microglia-like cells and/or microglia-precursor cells.

As used herein, a “carrier” refers to a surface enabling the adherence of cells thereto. Commonly, said surface may be, for example, the wall or bottom of a culture vessel, a plastic or glass slide such as for example a microscope slide or bead or membrane offering a surface for adherence. However, it is preferred that the wall or bottom of a culture vessel is not used as a carrier for practicing the present invention. It is rather preferred that a carrier used for practicing the present invention can float freely within cell culture medium.

As used herein, a “macrocarrier” differs from a microcarrier by its size and its shape. While microcarriers have a size in the micrometer range (e.g. 100 to 400 µm) and are typically spherical or cylindrical in shape, the macrocarriers usable in the present invention are generally larger (at least 0.1 cm (= 1 mm)) in at least one dimension, preferably in two dimensions, and are generally flat. This means the macrocarriers usable in the present invention can be described as sheets or membranes or films. The two-dimensional shape of the macrocarriers of the invention is not particularly limited: it could be a circle, a square, a rectangle, an oval, a polygon or any regular or irregular two-dimensional shape. There is no theoretical upper limit for the size of a macrocarrier usable in the present invention. In principle, a macrocarrier could be several meters long. In practice, however, the upper size limit of the macrocarrier is determined by the size of the culture flasks or the bioreactor.

As used herein, a “mesh membrane” refers to a permeable membrane, having an open weave with openings through which liquids (especially aqueous solutions) can pass, formed as a woven material, a perforated material or the like. The mesh membrane can be shaped as a lattice with square or rectangular or hexagonal openings.

As used herein, the term “about” encompasses the explicitly recited amounts as well as deviations therefrom of ± 10%. More preferably, a deviation of 5% is encompassed by the term “about”.

In the context of the present invention, the term “long-term cell differentiation” refers to a cell differentiation protocol, in which the cells are cultivated for a duration of 4 weeks or more, e.g., for a duration between 4 weeks and 1 year, between 4 weeks and 11 months, between 4 weeks and 10 months, between 4 weeks and 9 months, between 4 weeks and 8 months, between 4 weeks and 7 months, between 4 weeks and 6 months, between 4 weeks and 5 months, between 4 weeks and 4 months, between 4 weeks and 3 months, or between 4 weeks and 2 months.

As used herein, the expression “cryopreservable” when used in reference to a cell population means that at least 50% of the cells can be recovered after cryopreservation. In other words, when comparing the concentration (or number) of viable cells before and after the cryopreservation procedure, then the concentration (or number) of viable cells after the cryopreservation procedure is at least 50% of the corresponding concentration (or number) of viable cells before the the cryopreservation procedure.

Methods for determining the concentration or percentage of viable cells can be manual, semi-automated or automated, for example as described in Louis and Siegel (2011) and Cadena-Herrera et al (2015).

Media and methods known for cryopreservation of mammalian cells can be applied such as, but not restricted to, described in Li et al (2010) and Jang et al (2017). In addition, use of cryoprotectants such as anti-oxidatants or apoptosis inhibitors (Ha et al 2016) further improves recovery of cells.

### Embodiments of the Invention

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

In a first aspect the present invention is directed to a method for providing structural support to embryoid bodies for adherence and outgrowth, said method comprising the following steps:
(a) providing embryoid bodies;
(b) placing the embryoid bodies onto a macrocarrier, thereby obtaining a macrocarrier with adherent embryoid bodies; and
(c) culturing the macrocarrier with the adherent embryoid bodies of step (b) in cell-culture medium.

In some embodiments of the first aspect, the method is used for long-term cell differentiation.

In some embodiments of the first aspect, the macrocarrier is porous.

In some embodiments of the first aspect, the macrocarrier is a mesh membrane.

In some embodiments of the first aspect, the macrocarrier comprises, essentially consists of or consists of at least one material selected from the group consisting of nylon, PET, polyethylene, polypropylene, polyesters, and combinations of any of these.

In some embodiments of the first aspect, the macrocarrier is coated with a material facilitating cell adherence.

In some embodiments of the first aspect, the material facilitating cell adherence is selected from the group consisting of an extracellular matrix component, a synthetic coating, and combinations thereof. In some embodiments, the extracellular matrix component is selected from the group consisting of fibronectin, laminin, vitronectin, Matrigel™ (BD Biosciences), hyaluronic acid, collagen, elastin, proteoglycans, non-proteoglycan polysaccharides, and combinations of any of these. Proteoglycans and non-proteoglycan polysaccharides include heparan sulphate, dextran, dextran sulphate and chondroitin sulphate, among others. In some embodiments, synthetic coatings are selected from the group consisting of poly-lysine, poly-ornithine, polyethylenimine, biocompatible silicone, and combinations of any of these.

In some embodiments of the first aspect, the macrocarrier has a pore size in the range from about 5 to about 180 µm, preferably from about 10 µm to about 150 µm, more preferably from about 20 to about 100 µm, even more preferably from about 30 to about 80 µm, and most preferably from about 50 to about 70 µm.

In some embodiments of the first aspect, the size of the macrocarrier is at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm, in at least one dimension (i.e. length).

In some embodiments of the first aspect, the size of the macrocarrier is at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm, in two dimensions (i.e. length and width).

In some embodiments of the first aspect, the macrocarrier is a disk with a diameter of at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm.

There is no theoretical limit for the size of a macrocarrier usable in the present invention. In principle, a macrocarrier could be several meters long. In practice, however, the upper size limit of the macrocarrier is determined by the size of the culture flasks or the bioreactor. In preferred embodiments of the first aspect, the size of the macrocarrier is thus 30 cm or less, preferably 25 cm or less, more preferably 20 cm or less; more preferably 15 cm or less, even more preferably 10 cm or less, even more preferably 8 cm or less, in its largest dimension.

In some embodiments of the first aspect, the macrocarrier has a thickness in the range from 20 to 200 µm, preferably in the range from 40 to 150 µm, more preferably in the range from 50 to 135 µm.

In some embodiments of the first aspect, the embryoid bodies are placed onto the macrocarrier at a density from about 1 to about 70 embryoid bodies per cm², preferably from about 10 to about 65 embryoid bodies per cm², more preferably from about 20 to about 60 embryoid bodies per cm², even more preferably from about 30 to about 50 embryoid bodies per cm².

In some embodiments of the first aspect, the macrocarrier floats freely in the cell-culture medium.

In some embodiments of the first aspect, the cell culture medium is used according to a chosen differentiation protocol (e.g. a protocol disclosed by Abud et al., 2017; Muffat et al., 2016; or Haenseler et al., 2017) or as described in the second aspect below.

In some embodiments of the first aspect, the cell culture medium is precursor selection medium comprising
(1) a growth factor selected from the group consisting of 0.1 µM - 4.6 µM insulin and insulin-like growth factors;
(2) 5 to 100 ng/ml ligand stimulating CSF-receptor (preferably 10 to 75 ng/ml ligand, more preferably 15 to 50 ng/ml ligand, even more preferably 20 to 30 ng/ml ligand, and most preferably 25 ng/ml ligand); and
(3) 5 to 100 ng/ml IL3 (preferably 10 to 75 ng/ml IL3, more preferably 15 to 50 ng/ml IL3, even more preferably 20 to 30 ng/ml IL3, and most preferably 25 ng/ml IL3).
The ligand stimulating CSF receptor can be IL34, MCSF, or combinations thereof.

In a preferred embodiment of the first aspect, placed EBs according to step (b) are left/kept for a sufficient time, e.g. for at least 24 hours, to allow firm adherence of the embryoid bodies to the macrocarrier in static culture.

In some embodiments of the first aspect, the macrocarrier is subjected to dynamic movement during step (c) after firm adherence of EBs following step (b).

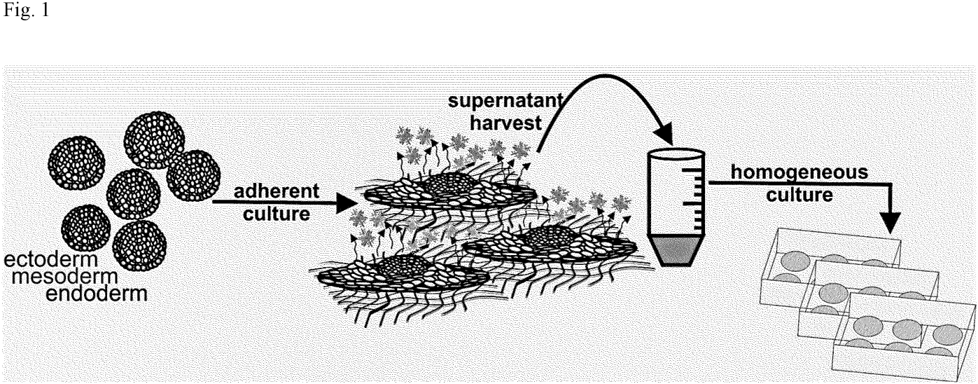

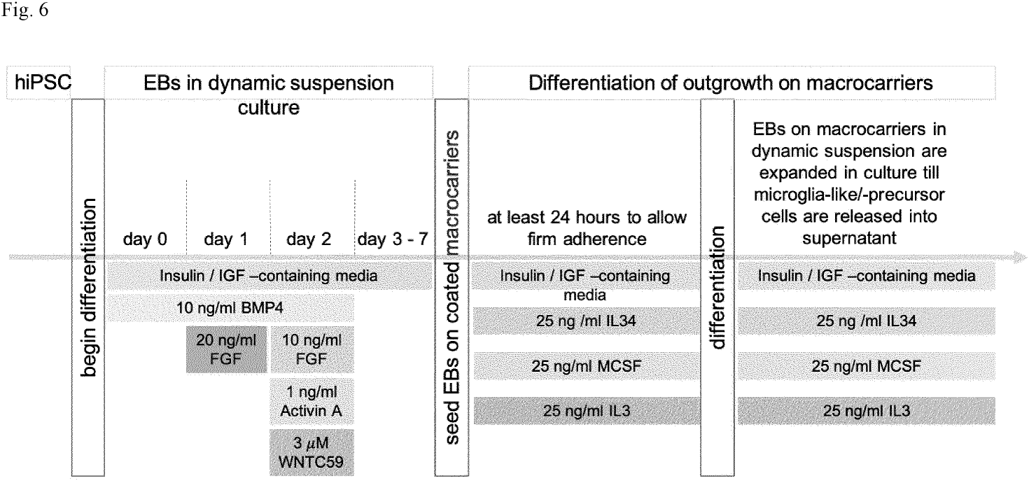

Different protocols for the differentiation of embryoid bodies to microglial precursor cells and/or microglia-like cells are known in the art. An overview of such protocols can be found in Timmerman R. et al. “An Overview of in vitro Methods to Study Microglia” Frontiers in Cellular Neuroscience, August 2018, volume 12, Article 242, pages 1-12, the content of which is hereby incorporated by reference; particular reference is made to FIG. 1 on page 6 of Timmerman et al., which provides a graphic overview of five different protocols. Any protocol from the prior art for the differentiation of embryoid bodies to microglial precursor cells and/or microglia-like cells is suitable for practicing the first aspect of the invention. Particularly well-suited protocols include the protocols described in (Abud et al., 2017; Haenseler et al., 2017; Muffat et al., 2016; Douvaras et al., 2017a; Pandya et al., 2017) and the protocol described in WO 2010/125110 A1, all of which are incorporated herein by reference. A novel protocol that is well-suited for practicing the present inventon is shown in FIG. 6.

In some embodiments of the first aspect, the embryoid bodies provided in step (a) are static-generated or dynamically-generated embryoid bodies.

In some embodiments of the first aspect, the embryoid bodies provided in step (i) were obtained by either following above-mentioned published protocols or culturing stem cells in suspension for at least 3 days in differentiation media comprising
(1) 0.1 to 50 ng/ml of an activator of TGFβ signaling (preferably 2.5 to 40 ng/ml of an activator of TGFβ signaling, more preferably 5 to 30 ng/ml of an activator of TGFβ signaling, even more preferably 7.5 to 20 ng/ml of an activator of TGFβ signaling, and most preferably 10 ng/ml of an activator of TGFβ signaling); and/or
(2) an activator of FGF signaling; and/or
(3) 0.03 to 15 µM of an inhibitor of WNT signaling (preferably 0.75 to 12 µM of an inhibitor of WNT signaling; more preferably 1.5 to 9 µM of an inhibitor of WNT signaling; even more preferably 2.25 to 6 µM of an inhibitor of WNT signaling; and most preferably 3 µM of an inhibitor of WNT signaling).

An activator of TGFβ signaling refers to any molecule or compound that activates TGFβ signaling. Activators of TGFβ signaling suitable to be used in the differentiation medium include, without limitation, proteins belonging to the TGFβ family, preferably BMP4 and Activin A and their functional analogs thereof.

An activator of FGF signaling refers to any molecule or compound that activates FGF signaling. Activators of FGF signaling suitable for use in the differentiation medium include, without limitation, ligands binding to FGF receptor such as FGF, preferably rhFGF, and aptamers specifically binding to the FGF receptor, such as the DNA aptamer TD0 (see Ueki et al. “DNA aptamer assemblies as fibroblast growth factor mimics and their application in stem cell culture” Chemical Communications 2019, 55, 2672-2675).

In some embodiments, 1 to 50 ng/ml rhFGF (preferably 2.5 to 40 ng/ml rhFGF, more preferably 5 to 30 ng/ml rhFGF, even more preferably 7.5 to 20 ng/ml rhFGF, and most preferably 10 ng/ml rhFGF) are used in the differentiation media.

An inhibitor of WNT signaling refers to any molecule or compound that inhibits WNT signaling. Inhibitors of WNT signaling suitable to be used in the differentiation medium include, without limitation, a protein belonging to the family of WNT-inhibitors, preferably WNT-C59 and functional analogs thereof.

In some embodiments of the first aspect, said induction of embryoid bodies comprises the following steps using medium comprising insulin/insulin-like growth factors:
(1) differentiating EBs for 1 day in a medium comprising 10 ng/ml BMP4; and
(2) further differentiating EBs for 1 day in a medium comprising 10 ng/ml BMP4 and 20 ng/ml rhFGF;
(3) further differentiating EBs for 1 day in a medium comprising 10 ng/ml BMP4, 10 ng/ml rhFGF, 1 ng/ml Activin A, and 3 µM WNT-C59; and
(4) optionally the differentiation of EBs can be extended for up to 5 days without the addition of additional factors.

Without wishing to be bound by any particular theory, the inventors believe that the presence of neurectodermal cells in the embryoid bodies is advantageous for practing the present invention. Embryoid bodies generated by the above protocols contain neurectodermal cells.

In a second aspect, the present invention is directed to a method for producing microglia-like cells and/or microglial precursor cells comprising the following steps:
(i) carrying out the method according to the first aspect (steps a, b, and c);
(ii) continuing cultivation of the macrocarrier with the adherent embryoid bodies in cell-culture medium until microglial precursor cells and/or microglial-like cells are released into the medium; and
(iii) optionally harvesting and/or cryopreserving the microglial precursor cells and/or microglia-like cells released into the medium.

In some embodiments of the second aspect, the method is used for long-term cell differentiation.

In some embodiments of the second aspect, the macrocarrier is subjected to dynamic movement during step (ii).

In one embodiment of the second aspect, the method for producing microglia-like cells and/or microglial precursor cells comprises the following step:
(i) carrying out the method according to the first aspect, wherein step (c) is carried out for at least 1 day(s), and wherein the cell-culture medium of step (c) is precursor selection medium comprising
   (1) a growth factor selected from the group consisting of 0.1 µM - 4.6 µM insulin and insulin-like growth factors;
   (2) 5 to 100 ng/ml ligand stimulating CSF-receptor (preferably 10 to 75 ng/ml ligand, more preferably 15 to 50 ng/ml ligand, even more preferably 20 to 30 ng/ml ligand, and most preferably 25 ng/ml ligand); and
   (3) 5 to 100 ng/ml IL3 (preferably 10 to 75 ng/ml IL3, more preferably 15 to 50 ng/ml IL3, even more preferably 20 to 30 ng/ml IL3, and most preferably 25 ng/ml IL3);
thereby generating neural cells and embryonic macrophage cells, and optionally endothelial-like cells.

The presence of neural cells can be confirmed by checking for the presence of nestin-positive cells; the presence of embryonic macrophage cells can be confirmed by checking for the presence of IBA1-positive cells; and the presence of endothelial-like cells can be confirmed by checking for CD31-positive cells.

The ligand stimulating CSF receptor can be IL34, MCSF, or combinations thereof.

In a further embodiment of the second aspect, the method for producing microglia-like cells and/or microglial precursor cells comprises the steps of:
(i) carrying out the method according to the first aspect, thereby obtaining a cell population comprising microglial and neural precursor cells;
(ii) continuing cultivation of the macrocarrier with the adherent embryoid bodies comprising precursor cells in medium comprising
   a growth factor selected from the group consisting of 0.1 µM - 4.6 µM insulin and insulin-like growth factors;
   5 to 100 ng/ml ligand stimulating CSF-receptor (preferably 10 to 75 ng/ml ligand, more preferably 15 to 50 ng/ml ligand, even more preferably 20 to 30 ng/ml ligand, and most preferably 25 ng/ml ligand); and
   5 to 100 ng/ml IL3 (preferably 10 to 75 ng/ml IL3, more preferably 15 to 50 ng/ml IL3, even more preferably 20 to 30 ng/ml IL3, and most preferably 25 ng/ml IL3); and
   thereby differentiating the cell population into microglial-like cells and obtaining expanded microglial precursor cells and/or microglia-like cells;
(iii) optionally harvesting the expanded microglial precursor cells and/or microglia-like cells; and
(iv) optionally maturing the expanded microglial precursor cells and/or microglia-like cells obtained in step (ii) or step (iii) for at least 24 hours in medium comprising
   a growth factor selected from the group consisting of 0.1 µM - 4.6 µM insulin and insulin-like growth factors;
   5 to 200 ng/ml ligand stimulating CSF-receptor (preferably 25 to 175 ng/ml ligand, more preferably 50 to 150 ng/ml ligand, even more preferably 75 to 125 ng/ml ligand, and most preferably 100 ng/ml ligand); and
   5 to 100 ng/ml TGFβ (preferably 20 to 80 ng/ml TGFβ, more preferably 30 to 70 ng/ml TGFβ, even more preferably 40 to 60 ng/ml TGFβ, and most preferably 50 ng/ml TGFβ.

As explained above, the ligand stimulating CSF receptor can be IL34, MCSF, or combinations thereof.

The protocol described in the preceding paragraph provides the advantage that cells can be matured in only 24 hours with the addition of IL34 and TGFβ. This is an improvement over the 2-week maturation protocols described in the prior art. Without wishing to be bound by any particular theory, the present inventors believe that this advantage is caused by providing a neural environment during steps (i) and (ii) of the second aspect. Such a neural environment is characterized by the presence of TUJ1-positive neural cells during steps (i) and (ii) of the above protocol.

In a third aspect the present invention is directed to a microglia-like cell or a microglial precursor cell obtainable or obtained by the method according to the second aspect.

In some aspects of the third aspect, the microglia-like cell and/or the microglial precursor cell is cryopreservable.

In a fourth aspect the present invention is directed to a use of a macrocarrier as structural support for adherence and outgrowth of embryoid bodies.

In some embodiments of the fourth aspect, the use comprises long-term cell differentiation.

In some embodiments of the fourth aspect, the macrocarrier is porous.

In some embodiments of the fourth aspect, the macrocarrier is a mesh membrane.

In some embodiments of the fourth aspect, the macrocarrier comprises, essentially consists of or consists of at least one material selected from the group consisting of nylon, PET, polyethylene, polypropylene, polyesters, and combinations of any of these.

In some embodiments of the fourth aspect, the macrocarrier is coated with an extracellular matrix component or synthetic coatings. In some embodiments, the extracellular matrix component is selected from the group consisting of fibronectin, laminin, vitronectin, Matrigel™ (BD Biosciences), hyaluronic acid, collagen, elastin, proteoglycans, non-proteoglycan polysaccharides, and combinations of any of these. - Proteoglycans and non-proteoglycan polysaccharides include heparan sulphate, dextran, dextran sulphate and chondroitin sulphate, among others. In some embodiments, synthetic coatings are selected from the group consisting of poly-lysine, poly-omithine, polyethylenimine, biocompatible silicone, and combinations of any of these.

In some embodiments of the fourth aspect, the macrocarrier has a pore size in the range from about 5 to about 180 µm, preferably from about 10 µm to about 150 µm, more preferably from about 20 to about 100 µm, even more preferably from about 30 to about 80 µm, and most preferably from about 50 to about 70 µm.

In some embodiments of the fourth aspect, the size of the macrocarrier is at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm, in at least one dimension (i.e. length).

In some embodiments of the fourth aspect, the size of the macrocarrier is at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm, in two dimensions (i.e. length and width).

In some embodiments of the fourth aspect, the macrocarrier is a disk with a diameter of at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm.

As explained above in the context of the first aspect, there is no theoretical upper limit for the size of a macrocarrier usable in the present invention. However, in preferred embodiments of the fourth aspect, the size of the macrocarrier is 30 cm or less, preferably 25 cm or less, more preferably 20 cm or less; more preferably 15 cm or less, even more preferably 10 cm or less, even more preferably 8 cm or less, in its largest dimension.

In some embodiments of the fourth aspect, the macrocarrier has a thickness in the range from 20 to 200 µm, preferably in the range from 40 to 150 µm, more preferably in the range from 50 to 135 µm.

In some embodiments of the fourth aspect, the embryoid bodies are seeded onto the macrocarrier at a density from about 1 to about 70 embryoid bodies per cm², preferably from about 10 to about 65 embryoid bodies per cm², more preferably from about 20 to about 60 embryoid bodies per cm², even more preferably from about 30 to about 50 embryoid bodies per cm².

In some embodiments of the fourth aspect, the macrocarrier floats freely in the cell-culture medium.

In some embodiments of the fourth aspect, the cell culture medium is a differentiation medium comprising a growth factor selected from the group consisting of insulin and insulin-like growth factors; 5 to 100 ng/ml IL34; and 5 to 100 ng/ml IL3; and 5 to 100 ng/ml MCSF.

In some embodiments of the fourth aspect, the macrocarrier comprises adherent embryoid bodies.

In some embodiments of the fourth aspect, the macrocarrier is subjected to dynamic movement.

In some embodiments of the fourth aspect, the macrocarrier comprises adherent embryoid bodies and is subjected to dynamic movement.

In some embodiments of the fourth aspect, the embryoid bodies are static-generated or dynamically-generated embryoid bodies.

In a fifth aspect the present invention is directed to a macrocarrier coated with a material facilitating cell adherence, wherein said macrocarrier is porous.

In some embodiments of the fifth aspect, the macrocarrier is a mesh membrane.

In some embodiments of the fifth aspect, the macrocarrier comprises, essentially consists of or consists of at least one material selected from the group consisting of nylon, PET, polyethylene, polypropylene, polyesters, and combinations of any of these.

In some embodiments of the fifth aspect, the material facilitating cell adherence is selected from the group consisting of an extracellular matrix component, a synthetic coating, and combinations thereof.

In some embodiments of the fifth aspect, the extracellular matrix component is selected from the group consisting of fibronectin, laminin, vitronectin, Matrigel™ (BD Biosciences), hyaluronic acid, collagen, elastin, proteoglycans, non-proteoglycan polysaccharides, and combinations of any of these. - Proteoglycans and non-proteoglycan polysaccharides include heparan sulphate, dextran, dextran sulphate and chondroitin sulphate, among others. In some embodiments of the fifth aspect, synthetic coatings are selected from the group consisting of poly-lysine, poly-omithine, polyethylenimine, biocompatible silicone, and combinations of any of these.

In some embodiments of the fifth aspect, the macrocarrier has a pore size in the range from about 5 µm to about 180 µm, preferably from about 10 µm to about 100 µm, more preferably from about 20 to about 90 µm, even more preferably from about 30 to about 80 µm, most preferably from about 50 to about 70 µm.

In some embodiments of the fifth aspect, the size of the macrocarrier is at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm, in at least one dimension (i.e. length).

In some embodiments of the fifth aspect, the size of the macrocarrier is at least 0.1 cm, preferably at least 0.2 cm, more preferably at least 0.3 cm, more preferably at least 0.5 cm, even more preferably at least 1.0 cm, even more preferably at least 1.5 cm, even more preferably at least 2.0 cm, in two dimensions (i.e. length and width).

In some embodiments of the fifth aspect, the macrocarrier is a disk with a diameter of at least 0.1 cm, preferably at least 0.2 cm, more preferably at least 0.3 cm, more preferably at least 0.5 cm; even more preferably at least 1.0 cm), even more preferably at least 1.5 cm, even more preferably at least 2.0 cm.

As explained above in the context of the first aspect, there is no theoretical upper limit for the size of a macrocarrier usable in the present invention. However, in preferred embodiments of the fifth aspect, the size of the macrocarrier is 30 cm or less, preferably 25 cm or less, more preferably 20 cm or less; more preferably 15 cm or less, even more preferably 10 cm or less, even more preferably 8 cm or less, in its largest dimension.

In some embodiments of the fifth aspect, the macrocarrier has a thickness in the range from 20 to 200 µm, preferably in the range from 40 to 150 µm, more preferably in the range from 50 to 135 µm.

In a sixth aspect the present invention is directed to a microglia-like cell or microglial precursor cell population, wherein said population is cryopreservable.

In some embodiments of the sixth aspect, the cell population is obtainable by carrying out the method according to the second aspect.

### EXAMPLES

The following examples are put forth so as to provide those of ordinary skill in the art with a complete disclosure and description of how to make and use the compositions and methods of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees centigrade, and pressure is at or near atmospheric.

### Example 1

Here we describe a novel implementation of mesh structures, typically used for filtration, as macrocarrier scaffolds to differentiate macrophage and microglia/-like cells from iPSCs using an EB-based technique. The mesh membrane made of biologically compatible material such as nylon, PET, polyethylene, polypropylene, or polyesters, among others (minimum 0.1 cm diameter and 5 µm to 180 µm pore size) are coated with extracellular matrix components such as fibronectin, laminin, collagen, elastin, proteoglycans, and non-proteoglycan polysaccharides, among others. The unique use of these macrocarriers can be implemented for any EB-based differentiation protocol to generate macrophages, microglia and their precursor or progenitor cell types in a scalable bioreactor format. Importantly, the mesh structure provides linear support to the EB and its outgrowth in the centimeter range while being in a free-floating state. These parameters allow for efficient scalability with regards to volume-to-surface area culture ratio as well as optimum supply with nutrients. In addition, the fluidic movement in a bioreactor setup facilitates detachment of the cell of interest in to the supernatant from which they can be easily harvested with a high degree of purity. Importantly, the free-floating culture in a non-tissue culture-treated vessel prevents detached cells from adhering to the vessel surface, thereby facilitating better accessibility to the cells and consequently further increasing the yield of the harvested cells.

### Differentiation of hiPSCs to Microglia

Nowadays, culture of hiPSCs is common practice worldwide. As such, hiPSCs described here were cultured in a standard feeder-free manner using commercially available culture media, known to a person skilled in the art.

To initiate differentiation towards the first phase (FIG. 1), three-dimensional spheres called embryoid bodies (EBs) were generated in differentiation media according to the above described publications or International Application WO 2010/125110 A1 or according to the EB-generation described in current application. EBs were cultured in suspension with media changes according to the respective protocol under static or dynamic conditions.

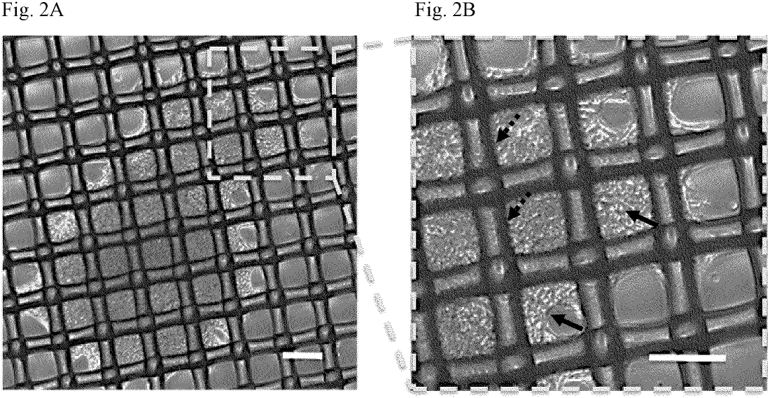

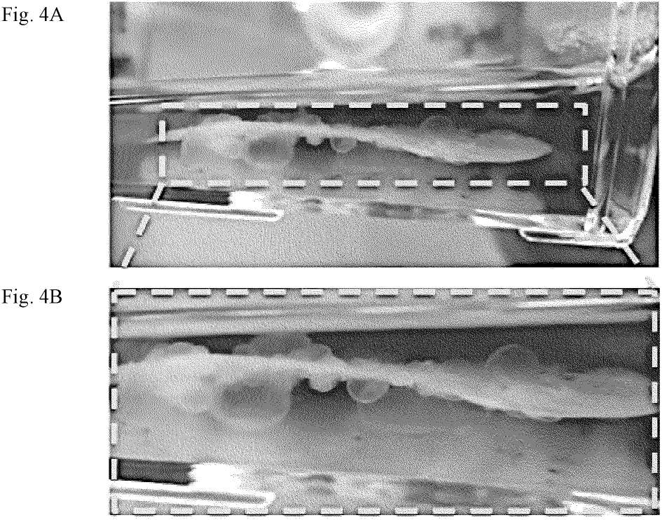

After EB induction, they were seeded onto extracellular matric (ECM)-coated sterile mesh membranes (MERCK Millipore, 47 mm diameter) at a density of up to 50 EBs per square centimeter, for up to 48 hours (usually only for up to 24 hours) to allow firm adherence. ECM coating was performed by submerging the macrocarriers in 1X phosphate-buffered saline (PBS) containing poly-L-ornithine (PLO) for 24 hours at 37° C. followed by submerging the macrocarriers in 1X PBS containing human fibronectin for 24 hours at 37° C. and finally storing the coated membranes in 1X PBS at 4° C. until used. Around twenty-four hours after seeding, the adherent EBs began to generate an outgrowth across and between the mesh pores (FIG. 2). Over time, as expected from hematopoietic iPSC differentiation protocols, cystic structures arose from the cellular outgrowth and were supported by the mesh membrane structure allowing for three-dimensional expansion (see FIGS. 4A and 4B).

Once added to the macrocarriers, the EBs were cultured in specification media according to chosen published protocol (for example (Abud et al., 2017; Haenseler et al., 2017) or according to WO 2010/125110 A1) or as described in current application.

The protocol used in the present application was based on the protocol of WO 2010/125110 A1 with some modifications, as described above in one preferred embodiment of the second aspect (see section “Detailed Description of the Invention” above) and as summarized in FIG. 6. The adherent EBs on macrocarriers were placed in dynamic culture for example, in a bioreactor setup. The EBs that were adherently attached on the macrocarriers generated adherent cellular outgrowths and covered the surface of the carriers with cells growing over the cross hairs (FIG. 2; zoom image, dotted arrows) as well as between the pores (FIG. 2; zoom image, solid line arrows). The macrocarriers, carrying the adherent EB outgrowths, were placed in free-floating culture formats, such as WAVE bioreactor system from GE healthcare or CERO bioreactor system from OLS. Accordingly, the dynamic movement of the bioreactor system, such as wave-like rocking motion of the WAVE bioreactor system or the bi-directional rotation of the CERO bioreactor system, caused the free-floating macrocarriers to flow repeatedly and consistently along the fluidic movement generated.

Once they were generated, these cells were released from the adherent differentiation culture into the supernatant as free-floating cells. The macrophage-, microglial-like or precursor cells in suspension were identified by phase-contrast microscopy as round-shiny cells of approximately 25 µm diameter. For experimentation, the harvested supernatant containing free-floating macrophage-, microglial-like or precursor cells can be collected, centrifuged to obtain cell pellet and plated as desired onto tissue culture formats in respective culture media according to the implemented protocol. In addition, cells can be mechanically purified by passing through a cell strainer ranging from 20 to 100 µm (most preferably 40 to 70 µm) before centrifugation.

The microglial-like/precursor cells obtained by the above method were surprisingly well-suited for cryopreservation. The cells harvested as described above were pelleted and resuspended in typical cryopreservation media, for example containing 90% knock-out serum replacement and 10% dimethyl sulfoxide. After thawing, at least 50% cell recovery has been observed. On the other hand, cells generated from currently used protocols such as Haenseler et al., 2017, were observed to only achieve up to 20% cell recovery using the same standard freezing technique.

### REFERENCES

The content of the following publications is incorporated herewith by reference in their entirety.
Abud, E.M., Ramirez, R.N., Martinez, E.S., Healy, L.M., Nguyen, C.H.H., Newman, S.A., Yeromin, A. V., Scarfone, V.M., Marsh, S.E., Fimbres, C., et al. (2017). iPSC-Derived Human Microglia-like Cells to Study Neurological Diseases. Neuron 94, 278-293.e9.
Badenes, S.M., Fernandes, T.G., Rodrigues, C.A.V., Diogo, M.M., and Cabral, J.M.S. (2016). Microcarrier-based platforms for in vitro expansion and differentiation of human pluripotent stem cells in bioreactor culture systems. J. Biotechnol. 234, 71-82.
Bennetta, M.L., Bennetta, C., Liddelowa, S.A., Ajami, B., Zamanian, J.L., Fernhoff, N.B., Mulinyawe, S.B., Bohlen, C.J., Adil, A., Tucker, A., et al. (2016). New tools for studying microglia in the mouse and human CNS. Pnas 1525528113-.
Blasi, E., Barluzzi, R., Bocchini, V., Mazzolla, R., and Bistoni, F. (1990). Immortalization of murine microglial cells by a v-raf / v-myc carrying retrovirus. J. Neuroimmunol. 27, 229-237.
Bosshart, H., and Heinzelmann, M. (2016). THP-1 cells as a model for human monocytes. Ann. Transl. Med. 4, 4-7.
Ditadi, A., and Sturgeon, C.M. (2016). Directed differentiation of definitive hemogenic endothelium and hematopoietic progenitors from human pluripotent stem cells. Methods 101, 65-72.
Douvaras, P., Sun, B., Wang, M., Kruglikov, I., Lallos, G., Zimmer, M., Terrenoire, C., Zhang, B., Gandy, S., Schadt, E., et al. (2017a). Directed Differentiation of Human Pluripotent Stem Cells to Microglia. Stem Cell Reports 8, 1516-1524.
Douvaras, P., Sun, B., MinghuiWang, Kruglikov, I., Lallos, G., Zimmer, M., Terrenoire, C., Zhang, B., Gandy, S., Schadt, E., et al. (2017b). Directed Differentiation of Human Pluripotent Stem Cells to Microglia. Stem Cell Reports 8, 1-9.
Galatro, T.F., Holtman, I.R., Lerario, A.M., Vainchtein, I.D., Brouwer, N., Sola, P.R., Veras, M.M., Pereira, T.F., Leite, R.E.P., Moller, T., et al. (2017). Transcriptomic analysis of purified human cortical microglia reveals age-associated changes. Nat. Neurosci. 20, 1162.
Galvão-Lima, L.J., Espindola, M.S., Soares, L.S., Zambuzi, F.A., Cacemiro, M., Fontanari, C., Bollela, V.R., and Frantz, F.G. (2017). Classical and alternative macrophages have impaired function during acute and chronic HIV-1 infection. Brazilian J. Infect. Dis. 21, 42-50.
Garcia-Reitboeck, P., Phillips, A., Piers, T.M., Villegas-Llerena, C., Butler, M., Mallach, A., Rodrigues, C., Arber, C.E., Heslegrave, A., Zetterberg, H., et al. (2018). Human Induced Pluripotent Stem Cell-Derived Microglia-Like Cells Harboring TREM2 Missense Mutations Show Specific Deficits in Phagocytosis. Cell Rep. 24, 2300-2311.
Ginhoux, F., Greter, M., Leboeuf, M., Nandi, S., See, P., Gokhan, S., Mehler, M.F., Conway, S.J., Ng, L.G., Stanley, E.R., et al. (2010). Fate Mapping Analysis Reveals That Adult Microglia Derive from Primitive Macrophages. Science (80-. ). 330, 841-845.
Gosselin, D., Link, V.M., Romanoski, C.E., Fonseca, G.J., Eichenfield, D.Z., Spann, N.J., Stender, J.D., Chun, H.B., Garner, H., Geissmann, F., et al. (2014). Environment drives selection and function of enhancers controlling tissue-specific macrophage identities. Cell 159, 1327-1340.
Gosselin, D., Skola, D., Coufal, N.G., Holtman, I.R., Schlachetzki, J.C.M., Sajti, E., Jaeger, B.N., O’Connor, C., Fitzpatrick, C., Pasillas, M.P., et al. (2017). An environment-dependent transcriptional network specifies human microglia identity. Science (80-. ). 356, 1248-1259.
Haenseler, W., Sansom, S.N., Buchrieser, J., Newey, S.E., Moore, C.S., Nicholls, F.J., Chintawar, S., Schnell, C., Antel, J.P., Allen, N.D., et al. (2017). A Highly Efficient Human Pluripotent Stem Cell Microglia Model Displays a Neuronal-Co-culture-Specific Expression Profile and Inflammatory Response. Stem Cell Reports 8, 1727-1742.
Iwasaki, H., Somoza, C., Shigematsu, H., Duprez, E.A., Iwasaki-Arai, J., Mizuno, S., Arinobu, Y., Geary, K., Zhang, P., Dayaram, T., et al. (2005). Distinctive and indispensable roles of PU.1 in maintenance of hematopoietic stem cells and their differentiation. Blood 106, 1590-1600.
Janabi, N., Peudenier, S., Heron, B., Ng, K.H., and Tardieu, M. (1995). Establishment of human microglial cell lines after transfection of primary cultures of embryonic microglial cells with the SV40 large T antigen. Neurosci. Lett. 195, 105-108.
Kierdorf, K., Erny, D., Goldmann, T., Sander, V., Schulz, C., Perdiguero, E.G., Wieghofer, P., Heinrich, A., Riemke, P., Hölscher, C., et al. (2013). Microglia emerge from erythromyeloid precursors via Pu.1-and Irf8-dependent pathways. Nat. Neurosci. 16, 273-280.
Kleiveland, C.R. (2015). Peripheral Blood Mononuclear Cells. In The Impact of Food Bioactives on Health: In Vitro and Ex Vivo Models, K. Verhoeckx, P. Cotter, I. López-Expósito, C. Kleiveland, T. Lea, A. Mackie, T. Requena, D. Swiatecka, and H. Wichers, eds. (Cham: Springer International Publishing), pp. 161-167.
Liu, W., Brosnan, C.F., Dickson, D.W., and Lee, S.C. (1994). Macrophage colony-stimulating factor mediates astrocyte-induced microglial ramification in human fetal central nervous system culture. Am. J. Pathol. 145, 48-53.
Medvinsky, A., Rybtsov, S., and Taoudi, S. (2011). Embryonic origin of the adult hematopoietic system: Advances and questions. Development 138, 1017-1031.
Muffat, J., Li, Y., Yuan, B., Mitalipova, M., Omer, A., Corcoran, S., Bakiasi, G., Tsai, L.-H., Aubourg, P., Ransohoff, R.M., et al. (2016). Efficient derivation of microglia-like cells from human pluripotent stem cells. Nat Med 22, 1358-1367.
Napoli, I. (2008). Establishment of Embryonic Stem Cell Derived Microglial Precursors and Application in an Animal Model of Alzheimer ’ s Disease.
Pandya, H., Shen, M.J., Ichikawa, D.M., Sedlock, A.B., Choi, Y., Johnson, K.R., Kim, G., Brown, M.A., Elkahloun, A.G., Maric, D., et al. (2017). Differentiation of human and murine induced pluripotent stem cells to microglia-like cells. Nat. Neurosci. 20, 753-759.
Prinz, M., and Priller, J. (2014). Microglia and brain macrophages in the molecular age: from origin to neuropsychiatric disease. Nat. Rev. Neurosci. 15, 300.
Ransohoff, R.M., and Perry, V.H. (2009). Microglial Physiology: Unique Stimuli, Specialized Responses. Annu. Rev. Immunol. 27, 119-145.
Sturgeon, C.M., Ditadi, A., Awong, G., Kennedy, M., and Keller, G. (2014). Wnt signaling controls the specification of definitive and primitive hematopoiesis from human pluripotent stem cells. Nat. Biotechnol. 32, 554-561.
Takata, K., Kozaki, T., Lee, C.Z.W., Thion, M.S., Otsuka, M., Lim, S., Utami, K.H., Fidan, K., Park, D.S., Malleret, B., et al. (2017). Induced-Pluripotent-Stem-Cell-Derived Primitive Macrophages Provide a Platform for Modeling Tissue-Resident Macrophage Differentiation and Function. Immunity 47, 183-198.e6.
Tsuchiya, T., Park, K.C., Toyonaga, S., Yamada, S.M., Nakabayashi, H., Nakai, E., Ikawa, N., Furuya, M., Tominaga, A., and Shimizu, K. (2005). Characterization of microglia induced from mouse embryonic stem cells and their migration into the brain parenchyma. J. Neuroimmunol. 160, 210-218.
van Wilgenburg, B., Browne, C., Vowles, J., and Cowley, S.A. (2013). Efficient, Long Term Production of Monocyte-Derived Macrophages from Human Pluripotent Stem Cells under Partly-Defined and Fully-Defined Conditions. PLoS One.
Louis K.S., Siegel A.C. (2011) Cell Viability Analysis Using Trypan Blue: Manual and Automated Methods. In: Stoddart M. (eds) Mammalian Cell Viability. Methods in Molecular Biology (Methods and Protocols), vol 740. Humana Press, pp. 7-12.
Cadena-Herrera D, Esparza-De Lara JE, Ramírez-Ibañez ND, López-Morales CA, Pérez NO, Flores-Ortiz LF, Medina-Rivero E. (2015). Validation of three viable-cell counting methods: Manual, semi-automated, and automated. Biotechnol Rep (Amst). 7:9-16.
Li Y, Tan J-C, Li L-S (2010). Comparison of three methods for cryopreservation of human embryonic stem cells. Fertility and Sterility, 93(3): 999-1005.
Jang TH, Park SC, Yang JH, Kim JY, Seok JH, Park US, Choi CW, Lee SR, Han J. (2017). Cryopreservation and its clinical applications. Integr Med Res. 6(1): 12-18.
Ha SJ, Kim BG, Lee YA, Kim YH, Kim BJ, Jung SE, Pang MG, Ryu BY. (2016). Effect of Antioxidants and Apoptosis Inhibitors on Cryopreservation of Murine Germ Cells Enriched for Spermatogonial Stem Cells. PLoS One. 77(8):e0161372.

1. A method for providing structural support to embryoid bodies for adherence and outgrowth, said method comprising the following steps:
(a) providing embryoid bodies;
(b) seeding the embryoid bodies onto a macrocarrier, thereby obtaining a macrocarrier with adherent embryoid bodies, wherein the microcarrier is porous; and
(c) culturing the macrocarrier with the adherent embryoid bodies of step (b) in cell-culture medium.

2. The method according to claim 1, wherein the macrocarrier has one or more of the following features:
(i) the macrocarrier is a mesh membrane;
(ii) the macrocarrier comprises, essentially consists of or consists of at least one material selected from the group consisting of nylon, PET, polyethylene, polypropylene, polyesters, and combinations of any of these;
(iii) the macrocarrier is coated with a material facilitating cell adherence;
(iv) the macrocarrier has a pore size in the range from about 5 to about 180 µm;
(v) the size of the macrocarrier is at least 0.1 cm in at least one dimension;
(vi) the macrocarrier is a disk with a diameter of at least 0.1 cm.

3. The method according to claim 1, wherein the embryoid bodies are seeded onto the macrocarrier at a density from about 1 to about 70 embryoid bodies per cm².

4. The method according to claim 1, wherein the macrocarrier floats freely in the cell-culture medium.

5. The method according to claim 1, wherein the macrocarrier is subjected to dynamic movement during step (c).

6. A method for producing microglia-like cells and/or microglial precursor cells comprising the following steps:
(i) carrying out the method according to claim 1 ;
(ii) continuing cultivation of the macrocarrier with the adherent embryoid bodies in cell-culture medium until microglial precursor cells and/or microglial-like cells are released into the medium; and
(iii) optionally harvesting and/or cryopreserving the microglial precursor cells and/or microglia-like cells released into the medium.

7. A microglia-like cell or microglial precursor cell obtainable by the method according to claim 6.

8. Use of a macrocarrier as structural support for adherence and outgrowth of embryoid bodies, wherein the macrocarrier is porous.

9. The use according to claim 8, wherein the macrocarrier has one or more of the following features:
(i) the macrocarrier is a mesh membrane;
(ii) the macrocarrier comprises, essentially consists of or consists of at least one material selected from the group consisting of nylon, PET, polyethylene, polypropylene, polyesters, and combinations of any of these;
(iii) the macrocarrier is coated with an extracellular matrix component or synthetic coatings, wherein preferably the extracellular matrix component is selected from the group consisting of fibronectin, laminin, vitronectin, Matrigel™, hyaluronic acid, collagen, elastin, proteoglycans, non-proteoglycan polysaccharides, and combinations of any of these, and wherein preferably synthetic coatings are selected from the group consisting of Poly-Lysine, Poly-Ornithine, Polyethylenimine, biocompatible silicone, and combinations of any of these;
(iv) the macrocarrier has a pore size in the range from about 5 to about 180 µm;
(v) the size of the macrocarrier is at least 0.1 cm in at least one dimension;
(vi) the macrocarrier is a disk with a diameter of at least 0.1 cm.

10. The use according to claim 8, wherein the embryoid bodies are seeded onto the macrocarrier at a density from about 1 to about 70 embryoid bodies per cm².

11. The use according to claim 8, wherein the macrocarrier floats freely in cell-culture medium.

12. The use according to claim 8 , wherein the macrocarrier comprises adherent embyoid bodies and is subjected to dynamic movement.

13. A macrocarrier coated with a material facilitating cell adherence, wherein said macrocarrier is porous.

14. The macrocarrier according to claim 13, wherein the macrocarrier has one or more of the following features:
(i) the macrocarrier is a mesh membrane;
(ii) the macrocarrier comprises, essentially consists of or consists of at least one material selected from the group consisting of nylon, PET, polyethylene, polypropylene, polyesters, and combinations of any of these;
(iii) the extracellular matrix component is selected from the group consisting of fibronectin, laminin, vitronectin, Matrigel™, hyaluronic acid, collagen, elastin, proteoglycans, non-proteoglycan polysaccharides, and combinations of any of these;
(iv) the synthetic coatings are selected from the group consisting of Poly-Lysine, Poly-Ornithine, Polyethylenimine, biocompatible silicone, and combinations of any of these;
(v) the macrocarrier has a pore size in the range from about 5 µm to about 180 µm;
(vi) the size of the macrocarrier is at least 0.1 cm;
(vii) the macrocarrier is a disk with a diameter of at least 0.1 cm.

15. A microglia-like cell population or microglial precursor cell population comprising the microglia-like cells or microglial precursor cells of claim 7.

16. The microglia-like cell population or microglial precursor cell population of claim 15, wherein said population is cryopreservable.
